(19) United States
(12) Patent Application Publication
(10) Pub. No.: US 2024/0304299 A1
(43) Pub. Date: Sep. 12, 2024

(54) INFORMATION PROCESSING SYSTEM, INFORMATION PROCESSING APPARATUS, AND INFORMATION PROCESSING METHOD

(71) Applicant: SONY GROUP CORPORATION, TOKYO (JP)

(72) Inventors: EIJIRO MORI, TOKYO (JP); RITSUKO KANO, TOKYO (JP)

(21) Appl. No.: 18/579,558

(22) PCT Filed: Mar. 2, 2022
(86) PCT No.: PCT/JP2022/008866

§ 371 Date: Jan. 16, 2024

(30) Foreign Application Priority Data
Jul. 27, 2021 (JP) 2021-122300

(51) Int. Cl. G16H 20/17

(52) U.S. Cl. CPC G16H 20/17

(57) ABSTRACT

An information processing system (10) according to an aspect of the present disclosure includes: an acquisition unit (21) that acquires medication action information regarding a medication action of a patient; an analysis unit (22) that analyzes the medication action information and captures a plurality of predetermined actions regarding medication execution; and a medication information generation unit (42) that recognizes the medication execution based on the plurality of predetermined actions and generates medication information regarding the medication execution.

### FIELD

The present disclosure relates to an information processing system, an information processing apparatus, and an information processing method.

### BACKGROUND

Eradication of asthma is difficult, and the purpose of treatment is to control symptoms (for example, control such as no attacks). In order to control symptoms, it is necessary to grasp and record daily symptoms and medication status. Note that, as a medicine for asthma, a liquid inhalant has a large effect. A pressurized inhaler, a nebulizer, or the like is used to inhale the liquid. A nebulizer atomizes the inhalant, allowing for more reliable inhalation than pressurized inhalers.

### CITATION LIST

### Patent Literature

Patent Literature 1: JP 2017-74197 A

### SUMMARY

### Technical Problem

However, currently, the patient's medication status is grasped and recorded by manual recording and reporting of the patient. In addition, not only in the treatment of asthma but also in the treatment of other diseases, the grasping and recording of the medication status are often performed by manual recording and reporting of the patient. Therefore, it is difficult to grasp the medication status of the patient.

Therefore, the present disclosure proposes an information processing system, an information processing apparatus, and an information processing method capable of easily grasping a medication status of a patient.

### Solution to Problem

An information processing system according to the embodiment of the present disclosure includes: an acquisition unit that acquires medication action information regarding a medication action of a patient; an analysis unit that analyzes the medication action information and captures a plurality of predetermined actions related to medication execution; and a generation unit that recognizes the medication execution based on the plurality of predetermined actions and generates medication information regarding the medication execution.

An information processing apparatus according to the embodiment of the present disclosure includes: an acquisition unit that acquires medication action information regarding a medication action of a patient; and an analysis unit that analyzes the medication action information and captures a plurality of predetermined actions related to medication execution.

An information processing method according to the embodiment of the present disclosure in which a computer is configured to: acquire medication action information regarding a medication action of a patient; and analyze the medication action information and capture a plurality of predetermined actions related to the medication execution.

### BRIEF DESCRIPTION OF DRAWINGS

FIG. 1 is a first diagram illustrating an example of a schematic configuration of an information processing system according to an embodiment of the present disclosure.

FIG. 2 is a second diagram illustrating an example of a schematic configuration of an information processing system according to an embodiment of the present disclosure.

FIG. 3 is a first diagram for explaining an example of a medication action of asthma according to an embodiment of the present disclosure.

FIG. 4 is a second diagram for explaining an example of a medication action of asthma according to an embodiment of the present disclosure.

FIG. 5 is a first diagram for explaining an example of an inhaler according to an embodiment of the present disclosure.

FIG. 6 is a first diagram for explaining an example of preparation and measurement according to an embodiment of the present disclosure.

FIG. 7 is a second diagram for explaining an example of preparation and measurement according to the embodiment of the present disclosure.

FIG. 8 is a second diagram for explaining an example of an inhaler according to an embodiment of the present disclosure.

FIG. 9 is a third diagram for explaining an example of an inhaler according to an embodiment of the present disclosure.

FIG. 10 is a fourth diagram for explaining an example of an inhaler according to an embodiment of the present disclosure.

FIG. 11 is a diagram illustrating an example of a schematic configuration of hardware according to an embodiment of the present disclosure.

### DESCRIPTION OF EMBODIMENTS

Hereinafter, embodiments of the present disclosure will be described in detail with reference to the drawings. Note that the systems, the apparatuses, the methods, and the like according to the present disclosure are not limited by the embodiments. In addition, in each of the following embodiments, basically the same parts are denoted by the same reference signs, and redundant description is omitted.

One or more embodiments (including examples and modifications) described below can each be implemented independently. On the other hand, at least some of the plurality of embodiments described below may be appropriately combined with at least some of other embodiments. The plurality of embodiments may include novel features different from each other. Therefore, the plurality of embodiments can contribute to solving different objects or problems, and can exhibit different effects.

The present disclosure will be described according to the following order of items.
1. Embodiments
1-1. Configuration Example of Information Processing System
1-2. Example of Medication Action of Asthma
1-3. Example of Preparation and Measurement
1-4. Configuration Example of Another Inhaler
1-5. Actions and Effects
2. Other Embodiments
3. Configuration Example of Hardware
4. Appendix

### 1. EMBODIMENTS

### 1-1. Configuration Example of Information Processing System

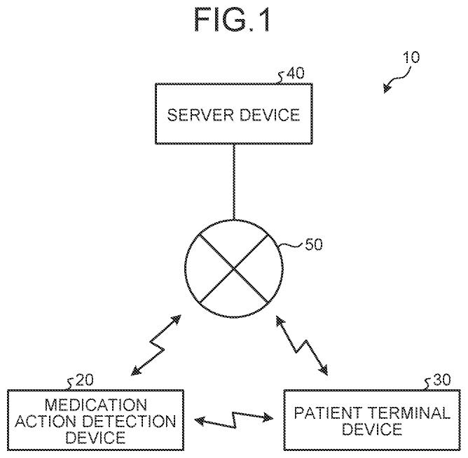

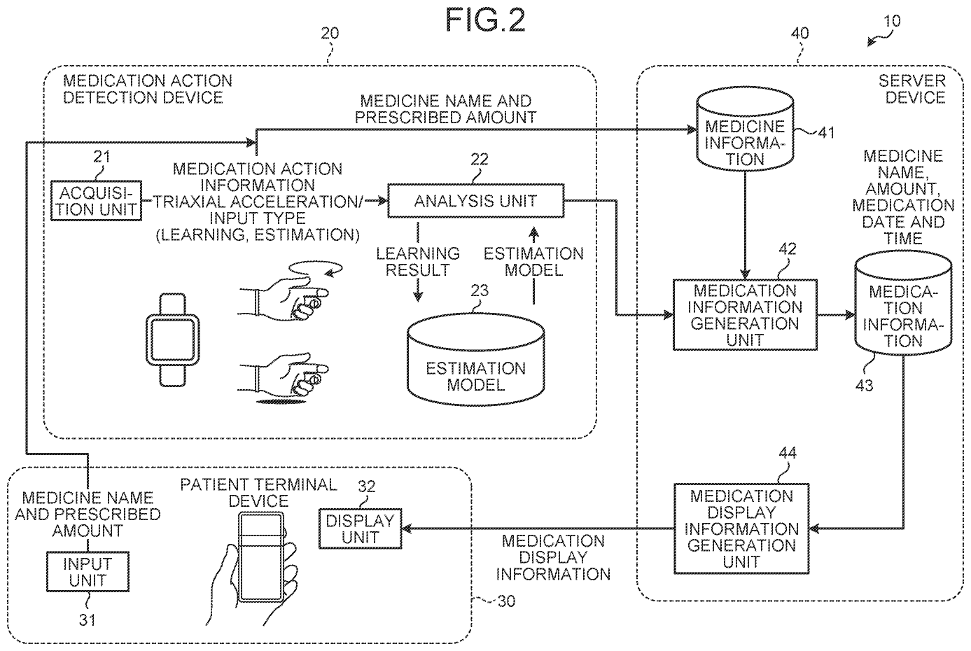

A configuration example of an information processing system 10 according to the present embodiment will be described with reference to FIGS. 1 and 2. FIGS. 1 and 2 are diagrams illustrating an example of a schematic configuration of the information processing system 10 according to the present embodiment.

As illustrated in FIG. 1, the information processing system 10 includes a medication action detection device 20, a patient terminal device 30, and a server device 40. The medication action detection device 20, the patient terminal device 30, and the server device 40 are communicably connected via both wireless and wired communication networks 50 or one of them. As the communication network 50, for example, the Internet, a wide area network (WAN), a local area network (LAN), a satellite communication network, or the like is used. Note that various types of information can be directly transmitted and received between the medication action detection device 20 and the patient terminal device 30 without the communication network 50.

As illustrated in FIG. 2, the medication action detection device 20 includes an acquisition unit 21, an analysis unit 22, and a storage unit 23. The medication action detection device 20 is used by a patient. As the medication action detection device 20, for example, a wristband-type wearable device or the like is used. As an example, the medication action detection device 20 is a smart watch, and the smart watch is attached to a wrist of a patient. Note that the medication action detection device 20 functions as an information processing apparatus.

The acquisition unit 21 acquires various types of information such as medication action information regarding the medication action of the patient. The acquisition unit 21 is realized by, for example, a sensor such as an acceleration sensor (as an example, a triaxial acceleration sensor). Note that the acquisition unit 21 may include a vital sensor capable of acquiring various vital data such as a pulse wave and sweating, a microphone, and the like.

Here, the medication action information includes, for example, a series of actions related to medication as the medication action. This series of actions includes, for example, actions on a container containing a medicine. The action on the container is, for example, an opening action of opening the container. In addition, among the series of actions, for example, there is a stationary action when taking a medicine.

In the learning stage, the analysis unit 22 constructs an estimation model, which is an example of a learning model, based on the medication action information acquired by the acquisition unit 21, and stores the estimation model in the storage unit 23. Note that the analysis unit 22 may update the estimation model based on the medication action information also in the estimation stage. As the estimation model, for example, a deep learning (DL) model, a convolutional neural network (CNN) model, or the like can be used.

Furthermore, in the estimation stage, the analysis unit 22 analyzes the medication action information acquired by the acquisition unit 21, and captures a plurality of predetermined actions related to medication by the patient. For example, the analysis unit 22 supplements a plurality of predetermined actions from the medication action information based on the estimation model stored in the storage unit 23. The analysis unit 22 supplements, for example, an opening action of opening the container and a stationary action when taking the medicine as the predetermined action, and generates analysis result information regarding the opening action and the stationary action. Thereafter, the analysis unit 22 transmits the generated analysis result information to the server device 40.

The storage unit 23 stores various types of information such as the estimation model obtained by the analysis unit 22. The storage unit 23 is realized by, for example, a storage device such as a flash memory or a random access memory (RAM). Note that various types of information obtained by the acquisition unit 21, the analysis unit 22, and the like are stored in the storage unit 23 as necessary.

The patient terminal device 30 includes an input unit 31 and a display unit 32. The patient terminal device 30 is used by the patient. For example, a smartphone or the like is used as the patient terminal device 30. The smartphone includes a camera, a microphone, an acceleration sensor, and the like.

The input unit 31 receives an input operation from an operator such as a patient. The input unit 31 is realized by, for example, an input device such as a touch panel or a button. Note that the input unit 31 may be realized by a voice input device (for example, a microphone or the like) that receives an input operation by a voice of the operator.

The display unit 32 displays various types of information. The display unit 32 is realized by, for example, a display device such as a liquid crystal display or an organic electro-luminescence (EL) display. Since various types of information are displayed by the display unit 32, the patient can visually recognize various types of information.

Here, the patient terminal device 30 transmits a plurality of pieces of input information input by the patient, for example, the name (medicine name) of the medicine to be taken, a prescribed amount (medicine prescribed amount) of the medicine, and the like to the medication action detection device 20. In addition, the medication action detection device 20 transmits the medication action information acquired by the acquisition unit 21 to the server device 40 together with the medicine name and the medicine prescribed amount received from the patient terminal device 30. Furthermore, the medication action detection device 20 transmits, to the server device 40, the analysis result information obtained by the analysis unit 22, that is, information regarding a plurality of predetermined actions related to medication.

As illustrated in FIG. 2, the server device 40 includes a medicine information storage unit 41, a medication information generation unit 42, a medication information storage unit 43, and a medication display information generation unit 44. The server device 40 functions as a management device. The server device 40 may be implemented by, for example, cloud computing.

The medicine information storage unit 41 stores medicine information transmitted from the patient terminal device 30. The medicine information includes, for example, information such as a medicine name, a prescribed amount, and medication action information. The medicine information storage unit 41 is realized by, for example, a storage device such as a flash memory, a RAM, or a hard disk.

The medication information generation unit 42 recognizes the medication execution and the medication amount based on the medicine information stored in the medicine information storage unit 41 and the analysis result information (for example, information regarding an opening action or a stationary action) transmitted from the medication action detection device 20, and generates medication information regarding the medication execution and the medication amount. Thereafter, the medication information generation unit 42 transmits the generated medication information to the medication information storage unit 43. The medication information includes, for example, information such as a medicine name, an amount (medication amount), and medication date and time.

The medication information storage unit 43 stores the medication information transmitted from the medication information generation unit 42. The medication information storage unit 43 is realized by, for example, a storage device such as a flash memory, a RAM, or a hard disk.

The medication display information generation unit 44 generates medication display information for displaying the medication information stored in the medication information storage unit 43, and transmits the medication display information to the patient terminal device 30. The patient terminal device 30 displays an image indicating the medication information by the display unit 32 based on the medication display information transmitted from the medication display information generation unit 44.

Here, each functional unit such as the acquisition unit 21, the analysis unit 22, the storage unit 23, the input unit 31, the display unit 32, the medicine information storage unit 41, the medication information generation unit 42, the medication information storage unit 43, and the medication display information generation unit 44 described above may be configured by both or either one of hardware and software. These configurations are not particularly limited. For example, each of the above-described functional units may be implemented by a computer such as a central processing unit (CPU) or a micro processing unit (MPU) executing a program stored in advance in a ROM using a RAM or the like as a work area. Furthermore, each functional unit may be realized by, for example, an integrated circuit such as an application specific integrated circuit (ASIC) or a field-programmable gate array (FPGA).

Note that the analysis unit 22 and the storage unit 23 described above are provided in the medication action detection device 20, but are not limited thereto, and may be provided in, for example, the patient terminal device 30 or the server device 40. In this case, the patient terminal device 30 and the server device 40 function as information processing apparatuses. At this time, a reception unit (not illustrated) included in the patient terminal device 30 or the server device 40 functions as an acquisition unit that receives and acquires the medication action information from the medication action detection device 20.

Furthermore, the medicine information storage unit 41, the medication information generation unit 42, the medication information storage unit 43, and the medication display information generation unit 44 are provided in the server device 40, but are not limited thereto, and may be provided in, for example, the patient terminal device 30 or the like. In this case, the patient terminal device 30 functions as a management device. Note that the patient terminal device 30 may be realized not only by a smartphone but also by a portable terminal such as a tablet, or may be realized by a personal computer or the like.

### 1-2. Example of Medication Action of Asthma

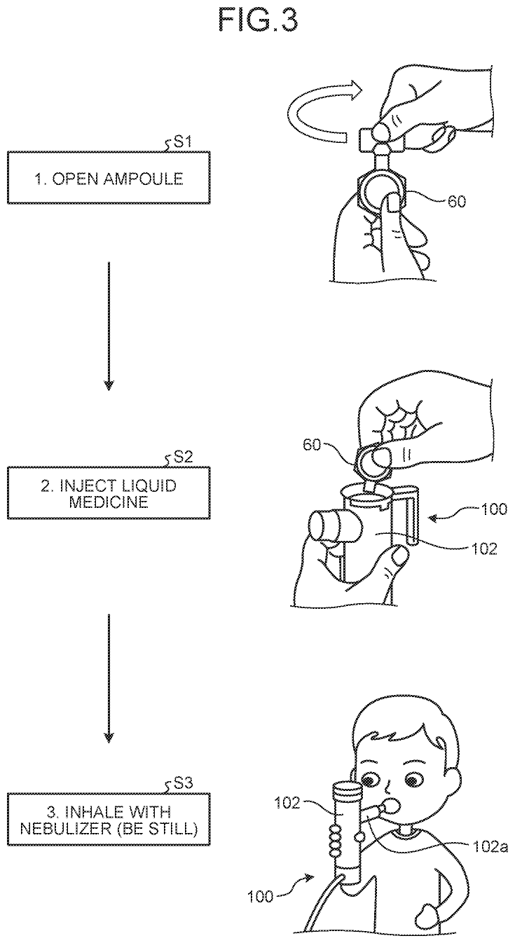

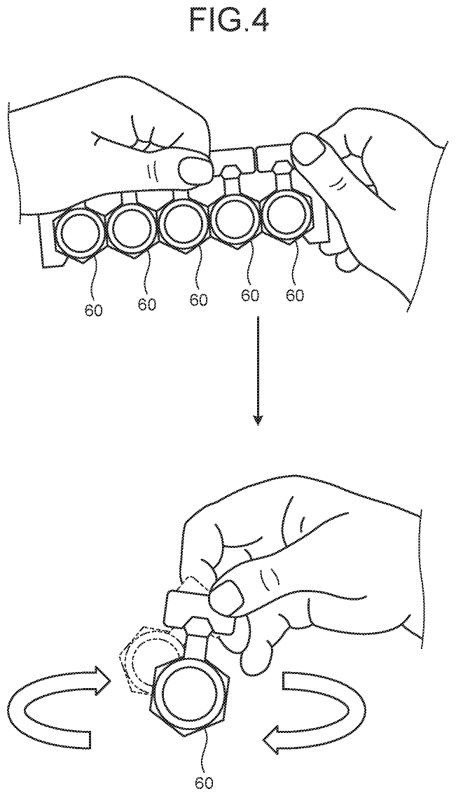

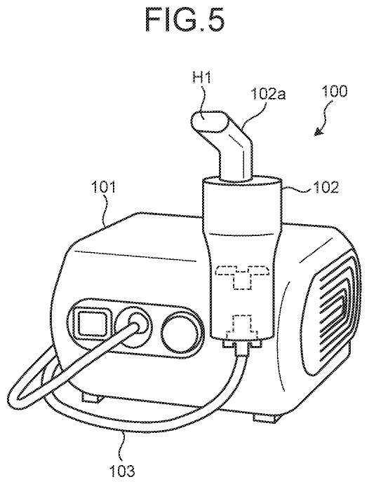

An example of a medication action of asthma according to the present embodiment will be described with reference to FIGS. 3 to 5. FIGS. 3 and 4 are diagrams for explaining an example of a medication action of asthma according to the present embodiment. FIG. 5 is a diagram for explaining an example of an inhaler 100 according to the present embodiment.

In the examples of FIGS. 3 and 4, the medication action detection device 20 is, for example, a smart watch. The smart watch is worn on the wrist of the patient. A patient wearing the smart watch takes a medicine for asthma (for example, liquid inhalants). At this time, the patient sits on a chair or the like to take medicine, and does not move parts other than the patient's hands and arms.

As illustrated in FIG. 3, the patient opens an ampoule 60 which is a container for storing the medicine (Step S1). Next, the patient injects a medicine from the ampoule 60 into an inhalation case 102 of the inhaler 100 (Step S2). Then, the patient holds a mouthpiece 102a of the inhalation case 102 still in his/her mouth, and inhales the medicine in the inhalation case 102 from the mouthpiece 102a (Step S3). After inhaling all the medicine, the patient releases his/her mouth from the mouthpiece 102a. Note that the inhaler 100 will be described later in detail.

The series of actions in Steps S1 to S3 is detected by the acquisition unit 21 of the medication action detection device 20, and medication action information is generated. Furthermore, before the actions in Steps S1 to S3 described above, for example, as illustrated in FIG. 4, the patient separates the ampoule 60 from the ampoule group, and then swings the ampoule 60 while holding the upper portion. The disengaging action, the swinging action, and the like may also be detected by the acquisition unit 21 of the medication action detection device 20 to generate the medication action information.

### (Inhaler)

As illustrated in FIG. 5, the inhaler 100 includes a device main body 101 and an inhalation case 102. The inhaler 200 is, for example, a compressor type nebulizer. The inhalation case 102 is connected to the device main body 101 by a tube 103. The inhalation case 102 is an example of a medication container. The device main body 101 atomizes the medicine in the inhalation case 102 and allows the patient to inhale the medicine. An upper portion of the inhalation case 102 is a mouthpiece 102a. The mouthpiece 102a has an inlet H1. Note that, in the example of FIG. 5, the inhaler 200 is a compressor type nebulizer, but is not limited thereto, and may be, for example, a mesh type nebulizer.

Here, in order to smoothly execute the actions in Steps S1 to S3 described above, for example, images, characters, and the like for explaining these actions may be displayed on the display unit 32 of the patient terminal device 30. For example, images of the actions illustrated in FIG. 3 (right side in FIG. 3) may be sequentially displayed on the display unit 32. As a result, a patient such as an elderly person or a child can visually recognize the medication action, so that the correct medication action can be smoothly performed.

Note that, in addition to the above-described medicine for asthma, for example, even in a case of taking a medicine such as eye drops or ear drops, it is possible to detect the medication action and generate the medication action information. Also in the medication action of eye drops or ear drops, there are actions in which a patient opens a cap of a container containing a medicine, brings the container to a position facing an eye or an ear, and drops the medicine from the container to the eye or the ear. This medication action includes an opening action and a stationary action. As described above, the medication action in the case of taking a medicine such as eye drops or ear drops may also be detected by the acquisition unit 21 of the medication action detection device 20, and the medication action information may be generated, similarly to the case of taking a medicine for asthma.

### 1-3. Example of Preparation and Measurement

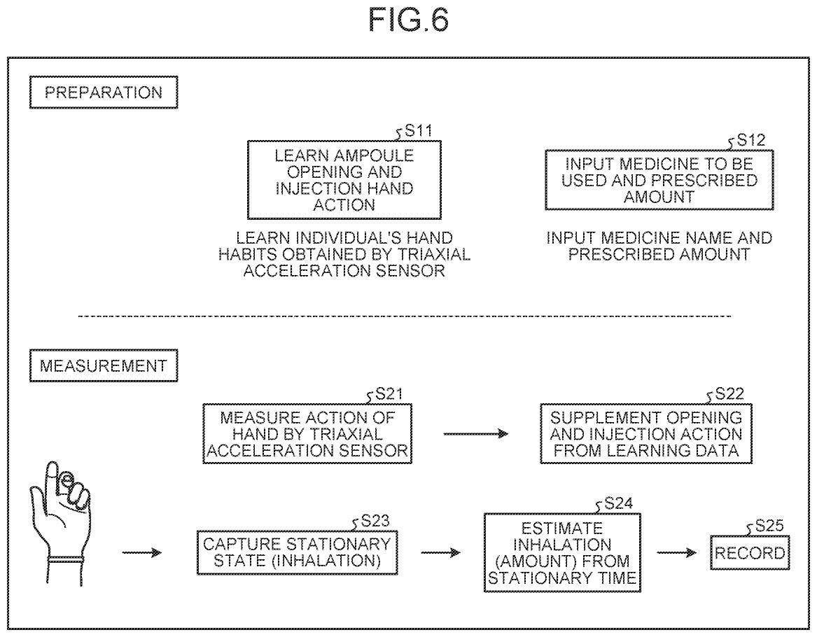

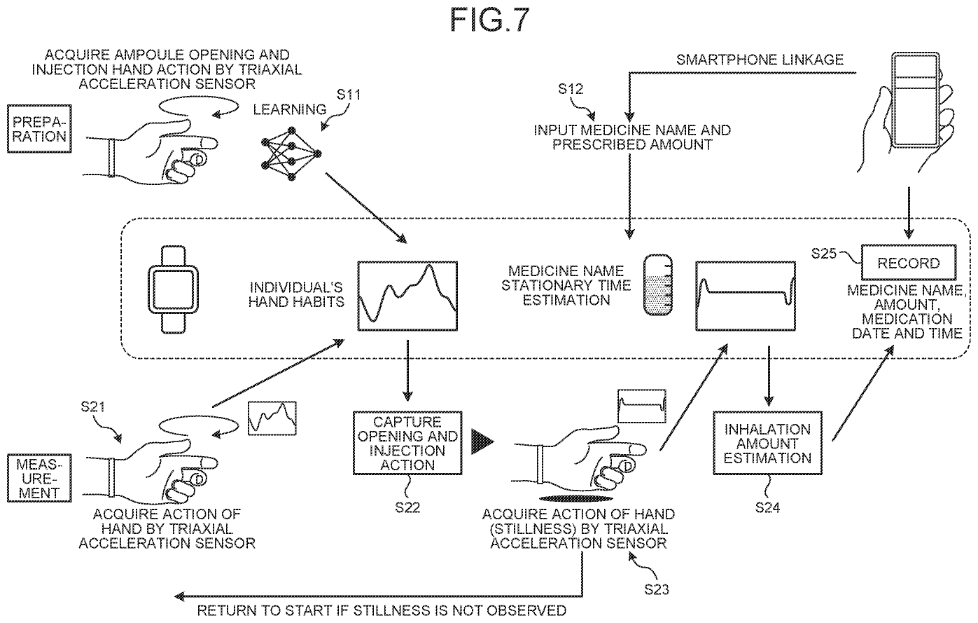

An example of preparation and measurement according to the present embodiment will be described with reference to FIGS. 6 and 7. FIGS. 6 and 7 are diagrams for explaining an example of preparation and measurement according to the present embodiment.

In the examples of FIGS. 6 and 7, the medication action detection device 20 is a smart watch, and the patient terminal device 30 is a smartphone. The smart watch is worn on the wrist of the patient, and the patient terminal device 30 is carried by the patient. A patient wearing a smart watch takes a medicine for asthma.

As illustrated in FIGS. 6 and 7, in the preparation stage, the acquisition unit 21 of the medication action detection device 20 detects the medication action of the patient and generates the medication action information, and the analysis unit 22 learns the medication action (for example, ampoule opening, injection hand action) of the individual patient based on the medication action information (Step S11). As a result, a learning model is constructed in the storage unit 23. This learning model is, for example, a personal learning model in which individual's hand habits or the like are learned.

In addition, the input unit 31 of the patient terminal device 30 inputs a medicine to be used (medicine name) and a prescribed amount according to an input operation from the patient (Step S12). The input medicine name and prescribed amount are transmitted to the server device 40 via the medication action detection device 20. The server device 40 stores the medicine information in the medicine information storage unit 41.

Next, in the measurement stage, the acquisition unit 21 of the medication action detection device 20 measures the medication action of the patient (for example, an action of a hand is measured by a triaxial acceleration sensor), and generates medication action information (Step S21). The analysis unit 22 supplements a plurality of predetermined actions, for example, an opening action which is opening and an injection action, based on the learning model (learning data) (Step S22). Furthermore, the analysis unit 22 supplements a stationary state (inhalation) as a stationary action, for example, based on the learning model (Step S23). The stationary action is an action (stop action) in which the patient brings each part such as his/her hand or arm into a stationary state.

Thereafter, the medication information generation unit 42 of the server device 40 recognizes the medication execution from the opening action, the injection action, and the stationary action, estimates the inhalation (inhalation amount) from the medicine information stored in the medicine information storage unit 41 and the stationary time of the stationary action (Step S24), generates the medication information regarding the medication execution and the inhalation amount, and stores the medication information in the medication information storage unit 43 (Step S25).

Here, regarding the estimation of the inhalation amount, for example, when the prescribed amount of the medicine in the container is known, the inhalation amount (medication amount) is obtained from the prescribed amount and the stationary time (inhalation time). For example, when the stationary time is longer than a predetermined time, it means that all the prescribed amount of medicine in the container has been inhaled. In addition to the prescribed amount, since the device performance can be known from the identification information such as the model number of the device, the inhalation amount can be obtained from the identification information and the stationary time. In this case, medicine information having identification information instead of the prescribed amount is stored in the medicine information storage unit 41 of the server device 40. Furthermore, the relationship between the stationary time and the inhalation amount may be obtained empirically or experimentally and stored in the medicine information storage unit 41 of the server device 40. In this case, the inhalation amount can be obtained from the stationary time based on the relationship information.

Note that, in Step S23 of FIG. 7, in a case where stillness is not observed, the processing returns to the first medication action detection processing. At this time, an image, a character, or the like indicating that stillness is not observed, that is, a stationary action is not detected may be displayed on the display unit 32 of the patient terminal device 30. As a result, the patient can grasp that the stationary action has not been detected.

In addition to the stationary action described above, in a case where the analysis unit 22 cannot recognize the action such as the opening action, the injection action, or the inhalation action in the preparation stage or the measurement stage, for example, in a case where the patient interrupts the action halfway or performs an action different from the normal action, an image, a character, or the like indicating the content of the action unrecognition may be displayed on the display unit 32 of the patient terminal device 30. As a result, a warning is issued to the patient, so that the patient can grasp a situation of action unrecognition.

According to the series of processing as described above, the medication action information is analyzed by the analysis unit 22, and a plurality of predetermined actions related to medication execution, for example, an opening action, an injection action, a stationary action, and the like are supplemented. Then, the medication execution and the medication amount are recognized by the medication information generation unit 42 based on the predetermined action, medication information regarding the medication execution and the medication amount is generated, and the medication information is stored in the medication information storage unit 43. In this way, since it is possible to obtain the medication information without requiring manual recording and reporting of the patient, it is possible to easily grasp the medication status of the patient. For example, by recognizing the medication execution and the medication amount based on a plurality of actions such as an opening action, an injection action, and a stationary action, it is possible to accurately recognize the medication execution and the medication amount as compared with a case where the medication execution and the medication amount are recognized based on one action, and thus, it is possible to accurately grasp the medication status of the patient.

Note that, as the predetermined action, for example, an opening action, an injection action, a stationary action, and the like are supplemented by the analysis unit 22, but the present invention is not limited thereto, and for example, other actions such as a disengaging action and a swinging action may be supplemented. In this case, by increasing the number of supplemented predetermined actions, it is possible to more accurately recognize the medication execution and the medication amount.

### 1-4. Configuration Example of Another Inhaler

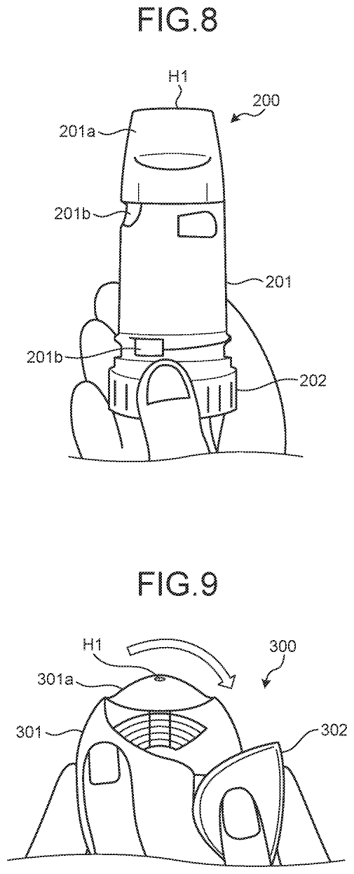

Examples of other inhalers 200, 300, and 400 according to the present embodiment will be described with reference to FIGS. 8 to 10. FIGS. 8 to 10 are diagrams for explaining examples of the inhalers 200, 300, and 400 according to the present embodiment, respectively.

As illustrated in FIG. 8, the inhaler 200 has a main body 201 and a rotary grip 202. The main body 201 accommodates a medicine. The rotary grip 202 is rotated by a predetermined amount by the patient to allow the patient to inhale the medicine. An upper portion of the main body 201 is a mouthpiece 201a, and a plurality of air ports 201b is formed on a side surface of the main body 201. The mouthpiece 201a has an inlet H1. Note that the main body 201 is detachably provided with a cap (not illustrated) covering an outer surface including the mouthpiece 201a.

With the inhaler 200 having such a configuration, the patient removes the cap from the main body 201 of the inhaler 200 and exposes the mouthpiece 201a located at the upper portion of the main body 201 (opening action). Then, the patient holds the main body 201 upright, and rotates the rotary grip 202 to allow the medicine in the main body 201 to be inhaled. For example, the patient turns the rotary grip 202 to the left and right by a predetermined amount to allow the medicine in the main body 201 to be inhaled. Thereafter, the patient stretches his/her back and exhales, and inhales the medicine in the main body 201 from the mouthpiece 201a located at the upper portion of the main body 201 (stationary action). After inhalation, the patient releases his/her mouth from a mouthpiece 401a and holds his/her breath for several seconds or more. Such a series of medication actions is detected by the medication action detection device 20, and medication action information is obtained by the acquisition unit 21. Note that the patient may repeat the action of inhaling the medicine as described above the number of times specified by the doctor.

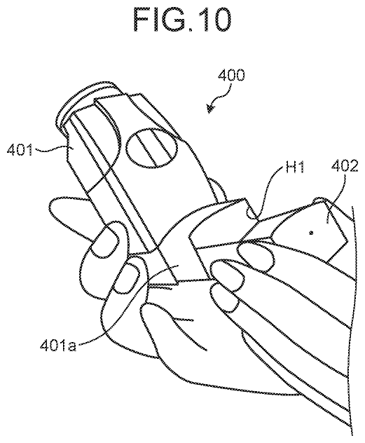

As illustrated in FIG. 9, the inhaler 300 has a main body 301 and a cover 302. The main body 301 accommodates a medicine. An upper portion of the main body 301 is a mouthpiece 301a. The mouthpiece 301a has an inlet H1. A cover 302 is provided on the main body 301 such that the inlet H1 of the mouthpiece 301a can be opened and closed.

With the inhaler 300 having such a configuration, the patient holds the inhaler 300 and opens the cover 302 of the inhaler 300 to expose the mouthpiece 301a located at the upper portion of the main body 301 (opening action). Then, the patient stretches his/her back and exhales, and inhales the medicine in the main body 301 from the mouthpiece 301a (stationary action). Thereafter, the patient releases his/her mouth from mouthpiece 401a and holds his/her breath for several seconds or more. Such a series of medication actions is detected by the medication action detection device 20, and medication action information is obtained by the acquisition unit 21. Note that the patient may repeat the action of inhaling the medicine as described above the number of times specified by the doctor.

As illustrated in FIG. 10, the inhaler 400 has a main body 401 and a cap 402. The main body 401 accommodates a medicine. A lower portion of the main body 401 is a mouthpiece 401a. The mouthpiece 401a has an inlet H1. A cap 402 is detachably attached to the mouthpiece 301a so as to cover the mouthpiece 401a.

With the inhaler 400 having such a configuration, the patient holds the inhaler 400, removes the cap 402 of the inhaler 400 from the main body 401, and exposes the mouthpiece 401a located at the lower portion of the main body 401 (opening action). Then, the patient stretches his/her back and exhales, and inhales the medicine in the main body 401 from the mouthpiece 401a (stationary action). Thereafter, the patient releases his/her mouth from mouthpiece 401a and holds his/her breath for several seconds or more. Such a series of medication actions is detected by the medication action detection device 20, and medication action information is obtained by the acquisition unit 21. Note that the patient may repeat the action of inhaling the medicine as described above the number of times specified by the doctor.

In any of the inhalers 200, 300, and 400 described above, the medication action information is obtained by the acquisition unit 21, the medication action information is analyzed by the analysis unit 22, and a plurality of predetermined actions related to medication execution, for example, an opening action, a stationary action, and the like are supplemented. Then, the medication execution and the medication amount are recognized by the medication information generation unit 42 based on the predetermined action, medication information regarding the medication execution and the medication amount is generated, and the medication information is stored in the medication information storage unit 43. In this way, since it is possible to obtain the medication information without requiring manual recording and reporting of the patient, it is possible to easily grasp the medication status of the patient.

### 1-5. Actions and Effects

As described above, according to the present embodiment, the information processing system 10 includes the acquisition unit 21 that acquires the medication action information regarding the medication action of the patient, the analysis unit 22 that analyzes the medication action information and captures a plurality of predetermined actions (for example, an opening action, a stationary action, or the like) regarding the medication execution, and the generation unit (for example, the medication information generation unit 42) that recognizes the medication execution based on the plurality of predetermined actions and generates the medication information regarding the medication execution. This makes it possible to obtain medication information without requiring manual recording and reporting of the patient, so that the medication status of the patient can be easily grasped.

Furthermore, the generation unit may obtain the medication amount of the patient based on a plurality of predetermined actions, and generate the medication information regarding the medication execution and the medication amount. As a result, it is possible to obtain the medication information regarding the medication execution and the medication amount, and thus, it is possible to more accurately grasp the medication status of the patient.

In addition, the generation unit may generate medication information including a medicine name taken by the patient, a medication date and time, and a medication amount. As a result, it is possible to obtain the medication information including the medicine name, the medication date and time, and the medication amount, and thus, it is possible to more accurately grasp the medication status of the patient.

In addition, the analysis unit 22 may capture a stationary action at the time of taking the medicine as one of the plurality of predetermined actions, and the generation unit may obtain the medication amount based on the stationary time of the stationary action. Accordingly, the medication amount can be easily obtained.

In addition, the generation unit may obtain the medication amount based on the stationary time of the stationary action and the prescribed amount of the medicine. This makes it possible to easily and accurately obtain the medication amount.

In addition, the stationary action may be a stationary action when inhaling a medicine. As a result, the inhalation amount can be easily obtained as the medication amount.

Furthermore, the medication information may include information regarding the inhalation amount of medicine as the medication amount. This makes it possible to obtain the medication information including the information regarding the inhalation amount.

Furthermore, the analysis unit 22 may capture a series of actions related to medication as a plurality of predetermined actions. As a result, the generation unit can recognize the medication execution based on a series of actions, and thus can accurately recognize the medication execution.

In addition, the analysis unit 22 may capture, as a plurality of predetermined actions, an opening action of opening a container (for example, ampoules 60, inhalers 200, 300, 400, and the like) containing a medicine and a stationary action when taking the medicine after the opening action. As a result, the generation unit can recognize the medication execution based on the opening action and the stationary action, and thus, can accurately recognize the medication execution.

In addition, the analysis unit 22 may capture, as the plurality of predetermined actions, an opening action of opening a container containing a medicine, an injection action of injecting the medicine in the container into the medication container, and a stationary action when taking the medicine after the injection action. As a result, the generation unit can recognize the medication execution based on the opening action, the injection action, and the stationary action, so that the medication execution can be more accurately recognized.

In addition, the medication action information may include an action on a container that stores a medicine as the medication action. As a result, the analysis unit 22 can recognize the action on the container that stores the medicine as the predetermined action.

In addition, the action on the container may be an opening action of opening the container. As a result, the analysis unit 22 can recognize the opening action of opening the container as the predetermined action.

In addition, the medication action information may include a stationary action when a medicine is taken as the medication action. As a result, the analysis unit 22 can recognize a stationary action when taking the medicine as the predetermined action.

In addition, the stationary action may be a stationary action when inhaling a medicine. As a result, the analysis unit 22 can recognize a stationary action when inhaling the medicine as the predetermined action.

Furthermore, the analysis unit 22 may generate a learning model for capturing a plurality of predetermined actions from the medication action information, and capture a plurality of predetermined actions from the medication action information based on the generated learning model. As a result, a plurality of predetermined actions can be easily and accurately captured from the medication action information.

Furthermore, the information processing system 10 may further include a storage unit (for example, the medication information storage unit 43) that stores medication information. Accordingly, the medication information can be easily managed.

Furthermore, the information processing system 10 may further include a display information generation unit (for example, the medication display information generation unit 44) that generates medication display information for displaying medication information. Accordingly, the medication display information can be obtained.

Furthermore, the information processing system 10 may further include a display unit 32 that displays an image indicating the medication information based on the medication display information. As a result, the user such as the patient can visually recognize the image indicating the medication information, and thus, can grasp the medication information.

### 2. Other Embodiments

The processing according to the above-described embodiments (or modifications) may be performed in various different modes (modifications) other than the above-described embodiments. For example, among the processes described in the above embodiments, all or a part of the processes described as being automatically performed can be manually performed, or all or a part of the processes described as being manually performed can be automatically performed by a known method. In addition, the processing procedure, specific name, and information including various data and parameters illustrated in the document and the drawings can be arbitrarily changed unless otherwise specified. For example, the various types of information illustrated in each figure are not limited to the illustrated information.

In addition, each component of each device illustrated in the drawings is functionally conceptual, and is not necessarily physically configured as illustrated in the drawings. That is, a specific form of distribution and integration of each device is not limited to the illustrated form, and all or a part thereof can be functionally or physically distributed and integrated in an arbitrary unit according to various loads, usage conditions, and the like.

In addition, the above-described embodiments (or modifications) can be appropriately combined within a range that does not contradict processing contents. Furthermore, the effects described in the present specification are merely examples and are not limited, and other effects may be provided.

### 3. Configuration Example of Hardware

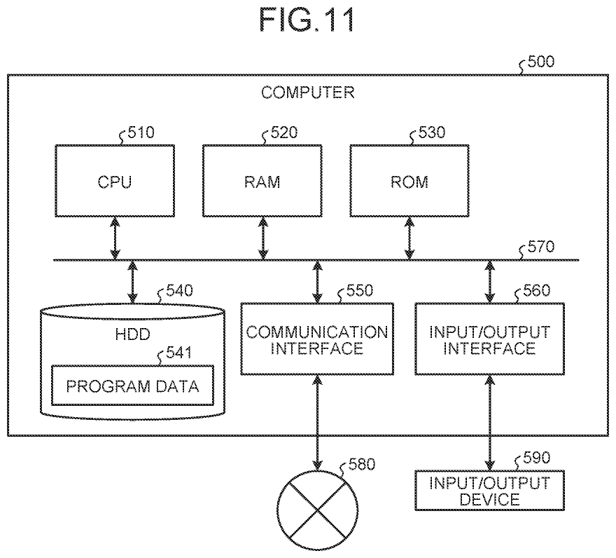

A specific hardware configuration example of the information device such as the medication action detection device 20, the patient terminal device 30, and the server device 40 according to the above-described embodiments (or modifications) will be described. The information device such as the medication action detection device 20, the patient terminal device 30, and the server device 40 according to the embodiments (or modifications) may be realized by, for example, a computer 500 having a configuration as illustrated in FIG. 11. FIG. 11 is a diagram illustrating a configuration example of hardware that realizes the function of the information device.

As illustrated in FIG. 11, the computer 500 includes a CPU 510, a RAM 520, a read only memory (ROM) 530, a hard disk drive (HDD) 540, a communication interface 550, and an input/output interface 560. Each unit of the computer 500 is connected by a bus 570.

The CPU 510 operates based on a program stored in the ROM 530 or the HDD 540, and controls each unit. For example, the CPU 510 develops a program stored in the ROM 530 or the HDD 540 in the RAM 520, and executes processing corresponding to various programs.

The ROM 530 stores a boot program such as a basic input output system (BIOS) executed by the CPU 510 when the computer 500 is activated, a program depending on hardware of the computer 500, and the like.

The HDD 540 is a computer-readable recording medium that non-transiently records a program executed by the CPU 510, data used by the program, and the like. Specifically, the HDD 540 is a recording medium that records an information processing program according to the present disclosure as an example of the program data 541.

The communication interface 550 is an interface for connecting the computer 500 to an external network 580 (for example, the Internet). For example, the CPU 510 receives data from another device or transmits data generated by the CPU 510 to another device via the communication interface 550.

The input/output interface 560 is an interface for connecting an input/output device 590 and the computer 500. For example, the CPU 510 receives data from an input device such as a keyboard and a mouse via the input/output interface 560. In addition, the CPU 510 transmits data to an output device such as a display, a speaker, or a printer via the input/output interface 560.

Note that the input/output interface 560 may function as a media interface that reads a program or the like recorded in a predetermined recording medium (medium). As the medium, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like is used.

Here, for example, in a case where the computer 500 functions as an information device such as the medication action detection device 20, the patient terminal device 30, the server device 40, or the like according to the embodiments, the CPU 510 of the computer 500 executes the information processing program loaded on the RAM 520 to implement all or some of the functions of each unit included in the information device. In addition, the HDD 540 stores an information processing program and data according to the present disclosure. Note that the CPU 510 reads and executes the program data 541 from the HDD 540, but as another example, these programs may be acquired from another device via the external network 580.

### 4. Appendix

Note that the present technology can also have the following configurations.
(1)

An information processing system comprising:
an acquisition unit that acquires medication action information regarding a medication action of a patient;
an analysis unit that analyzes the medication action information and captures a plurality of predetermined actions related to medication execution; and
a generation unit that recognizes the medication execution based on the plurality of predetermined actions and generates medication information regarding the medication execution.
(2)

The information processing system according to (1), wherein
the generation unit obtains a medication amount of the patient based on the plurality of predetermined actions, and generates the medication information regarding the medication execution and the medication amount.
(3)

The information processing system according to (2), wherein
the generation unit generates the medication information including a medicine name taken by the patient, a medication date and time, and the medication amount.
(4)

The information processing system according to (2) or (3), wherein
the analysis unit captures a stationary action when a medicine is taken as one of the plurality of predetermined actions, and
the generation unit obtains the medication amount based on a stationary time of the stationary action.
(5)

The information processing system according to (4), wherein
the generation unit obtains the medication amount based on the stationary time of the stationary action and a prescribed amount of the medicine.
(6)

The information processing system according to (4) or (5), wherein
the stationary action is a stationary action when the medicine is inhaled.
(7)

The information processing system according to (6), wherein
the medication information includes information regarding an inhalation amount of the medicine as the medication amount.
(8)

The information processing system according to any one of (1) to (7), wherein
the analysis unit captures a series of actions related to medication as the plurality of predetermined actions.
(9)

The information processing system according to any one of (1) to (8), wherein
the analysis unit captures, as the plurality of predetermined actions, an opening action of opening a container containing a medicine and a stationary action of taking the medicine after the opening action.
(10)

The information processing system according to any one of (1) to (8), wherein
the analysis unit captures, as the plurality of predetermined actions, an opening action of opening a container containing a medicine, an injection action of injecting the medicine in the container into a medication container, and a stationary action when taking the medicine after the injection action.
(11)

The information processing system according to any one of (1) to (10), wherein
the medication action information includes an action on a container that stores a medicine as the medication action.
(12)

The information processing system according to (11), wherein
the action on the container is an opening action of opening the container.
(13)

The information processing system according to any one of (1) to (12), wherein
the medication action information includes a stationary action when a medicine is taken as the medication action.
(14)

The information processing system according to (13), wherein
the stationary action is a stationary action when the medicine is inhaled.
(15)

The information processing system according to any one of (1) to (14), wherein
the analysis unit generates a learning model for capturing the plurality of predetermined actions from the medication action information, and captures the plurality of predetermined actions from the medication action information based on the learning model generated.
(16)

The information processing system according to any one of (1) to (15), further comprising
a storage unit that stores the medication information.
(17)

The information processing system according to any one of (1) to (16), further comprising
a display information generation unit that generates medication display information for displaying the medication information.
(18)

The information processing system according to (17), further comprising
a display unit that displays an image indicating the medication information based on the medication display information.
(19)

An information processing apparatus comprising:
an acquisition unit that acquires medication action information regarding a medication action of a patient; and
an analysis unit that analyzes the medication action information and captures a plurality of predetermined actions related to medication execution.
(20)

An information processing method in which
a computer is configured to:
acquire medication action information regarding a medication action of a patient; and
analyze the medication action information and capture a plurality of predetermined actions related to the medication execution.
(21)

An information processing method using the information processing system according to any one of (1) to (18).
(22)

An information processing apparatus related to the information processing system according to any one of (1) to (18).

### REFERENCE SIGNS LIST

10 INFORMATION PROCESSING SYSTEM
20 MEDICATION ACTION DETECTION DEVICE
21 ACQUISITION UNIT
22 ANALYSIS UNIT
23 STORAGE UNIT
30 PATIENT TERMINAL DEVICE
31 INPUT UNIT
32 DISPLAY UNIT
40 SERVER DEVICE
41 MEDICINE INFORMATION STORAGE UNIT
42 MEDICATION INFORMATION GENERATION UNIT
43 MEDICATION INFORMATION STORAGE UNIT
44 MEDICATION DISPLAY INFORMATION GENERATION UNIT
50 COMMUNICATION NETWORK
60 AMPOULE
100 INHALER
101 DEVICE MAIN BODY
102 INHALATION CASE
103 TUBE
102a MOUTHPIECE
200 INHALER
201 MAIN BODY
201a MOUTHPIECE
201b AIR PORT
202 ROTARY GRIP
300 INHALER
301 MAIN BODY
301a MOUTHPIECE
302 COVER
400 INHALER
401 MAIN BODY
401a MOUTHPIECE
402 CAP
H1 INLET

1. An information processing system comprising:
an acquisition unit that acquires medication action information regarding a medication action of a patient;
an analysis unit that analyzes the medication action information and captures a plurality of predetermined actions related to medication execution; and
a generation unit that recognizes the medication execution based on the plurality of predetermined actions and generates medication information regarding the medication execution.

2. The information processing system according to claim 1, wherein
the generation unit obtains a medication amount of the patient based on the plurality of predetermined actions, and generates the medication information regarding the medication execution and the medication amount.

3. The information processing system according to claim 2, wherein
the generation unit generates the medication information including a medicine name taken by the patient, a medication date and time, and the medication amount.

4. The information processing system according to claim 2, wherein
the analysis unit captures a stationary action when a medicine is taken as one of the plurality of predetermined actions, and
the generation unit obtains the medication amount based on a stationary time of the stationary action.

5. The information processing system according to claim 4, wherein
the generation unit obtains the medication amount based on the stationary time of the stationary action and a prescribed amount of the medicine.

6. The information processing system according to claim 4, wherein
the stationary action is a stationary action when the medicine is inhaled.

7. The information processing system according to claim 6, wherein
the medication information includes information regarding an inhalation amount of the medicine as the medication amount.

8. The information processing system according to claim 1, wherein
the analysis unit captures a series of actions related to medication as the plurality of predetermined actions.

9. The information processing system according to claim 1, wherein
the analysis unit captures, as the plurality of predetermined actions, an opening action of opening a container containing a medicine and a stationary action of taking the medicine after the opening action.

10. The information processing system according to claim 1, wherein
the analysis unit captures, as the plurality of predetermined actions, an opening action of opening a container containing a medicine, an injection action of injecting the medicine in the container into a medication container, and a stationary action when taking the medicine after the injection action.

11. The information processing system according to claim 1, wherein
the medication action information includes an action on a container that stores a medicine as the medication action.

12. The information processing system according to claim 11, wherein
the action on the container is an opening action of opening the container.

13. The information processing system according to claim 1, wherein
the medication action information includes a stationary action when a medicine is taken as the medication action.

14. The information processing system according to claim 13, wherein
the stationary action is a stationary action when the medicine is inhaled.

15. The information processing system according to claim 1, wherein
the analysis unit generates a learning model for capturing the plurality of predetermined actions from the medication action information, and captures the plurality of predetermined actions from the medication action information based on the learning model generated.

16. The information processing system according to claim 1, further comprising
a storage unit that stores the medication information.

17. The information processing system according to claim 1, further comprising
a display information generation unit that generates medication display information for displaying the medication information.

18. The information processing system according to claim 17, further comprising
a display unit that displays an image indicating the medication information based on the medication display information.

19. An information processing apparatus comprising:
an acquisition unit that acquires medication action information regarding a medication action of a patient; and
an analysis unit that analyzes the medication action information and captures a plurality of predetermined actions related to medication execution.

20. An information processing method in which
a computer is configured to:
acquire medication action information regarding a medication action of a patient; and
analyze the medication action information and capture a plurality of predetermined actions related to the medication execution.
